(19) United States
(12) Patent Application Publication
(10) Pub. No.: US 2020/0297035 A1
(43) Pub. Date: Sep. 24, 2020

(54) GLASS ATOMIZING CORE USED FOR ELECTRONIC CIGARETTE

(71) Applicants: Houqin Huang, Shenzhen (CN); Tipei Cai, Shenzhen (CN)

(72) Inventors: Houqin Huang, Shenzhen (CN); Tipei Cai, Shenzhen (CN)

(21) Appl. No.: 16/388,879

(22) Filed: Apr. 19, 2019

(30) Foreign Application Priority Data
Mar. 20, 2019 (CN) 201920351327.1

(51) Int. Cl. A24F 47/00; A61M 11/04; B05B 7/16

(52) U.S. Cl. CPC A24F 47/008; B05B 7/1686; A61M 11/042

(57) ABSTRACT

The invention relates to a glass atomizing core used for electronic cigarette, comprising a glass chamber, an atomizing bracket and an atomizing core arranged in sequence from outside to inside, an upper end of the glass chamber is clamped with an adduction silicone cover, a lower end thereof is arranged with a steel sleeve, an electrode is arranged on a lower end of the steel sleeve; the glass chamber is provided as a cannular shape, a concave notch is disposed on outer wall surface of a lower end thereof, a fixing ring is clamped with the notch, and the fixing ring is connected to the steel sleeve; the atomizing core is connected with a lower end of the atomizing bracket, an upper end of the atomizing core is wound with nonwovens, and a lower end thereof is sleeved with a ceramic silicone sleeve and electrically connected with the electrode.

### BACKGROUND OF THE INVENTION

### 1. Field of the Invention

The invention relates to the technical field of electronic products, in particular to a glass atomizing core used for electronic cigarette.

### 2. Background Art

At present, the prior atomizing cores of electronic cigarette are generally made of plastic materials, which can not withstand high temperature, and can be easily deformed and oil-leaked. In high temperatures, plastics may deteriorate and emit noxious gas, and in the process of consumption, the harmful substances may enter into human's body along with the smoke, therefore causing serious harm to human health. Moreover, the plastic atomizing core can carry a lower temperature than glass atomizing core, in use process, the repeat heating of plastic atomizing core may cause deformation or even rupture, the smoke oil may naturally overflow causing serious waste of resources, and the expensive smoke oil is not recyclable after overflowing; furthermore, the overflowing of the smoke oil also affect the environment.

### SUMMARY OF THE INVENTION

In order to solve the problems mentioned above, the invention provides the following technical schemes:

A glass atomizing core used for electronic cigarette, comprising a glass chamber, an atomizing bracket and an atomizing core arranged in sequence from outside to inside, wherein an upper end of the glass chamber is clamped with an adduction silicone cover,

a lower end of the glass chamber is arranged with a steel sleeve, and an electrode is arranged on a lower end of the steel sleeve;

the glass chamber is provided as a cannular shape, and a concave notch is disposed on outer wall surface of a lower end thereof, a fixing ring is clamped with the notch, and the fixing ring is connected to the steel sleeve;

the atomizing core is connected with a lower end of the atomizing bracket, an upper end of the atomizing core is wound with nonwovens, and a lower end of the atomizing core is sleeved with a ceramic silicone sleeve and electrically connected with the electrode.

Preferably, the glass chamber comprises a cylindrical portion I and a cylindrical portion II arranged in sequence from top to bottom, the outer diameter of the cylindrical portion I is larger than that of the cylindrical portion II, and the central axes of the two are collinear; a ring belt is arranged on the cylindrical portion II to form the notch adapted to the fixing ring.

Preferably, the fixing ring comprises a half-cylinder fixing ring I and a half-cylinder fixing ring II, a cylindrical fixing ring is composed of the fixing ring I and the fixing ring II; a clamping portion adopted to the ring belt is arranged on the inner wall of the fixing ring I and the fixing ring II, the clamping portion is provided as an annular groove structure.

Preferably, a chamber adopted to the atomizing core is provided in the atomizing bracket, the atomizing bracket comprises a circular-table portion I, a circular-table portion II, and a circular-table portion III, which are arranged in sequence from top to bottom, the outer diameters of the circular-table portion I, the circular-table portion II and the circular-table portion III are gradually increased, a transition portion is provided between the circular-table portion I and the circular-table portion II, and the central axes of the circular-table portion I, the circular-table portion II, the circular-table portion III and the transition portion are collinear.

Preferably, the circular-table portion II is provided with a groove at intervals, an O-shaped ring is provided in the groove, and an outer portion of the O-shaped ring is attached and connected with the lower end of the glass chamber; and a through hole is provided on the circular-table portion II.

Preferably, the chamber is arranged in the circular-table portion II and the circular-table portion III.

Preferably, a circular-table portion IV is arranged on a lower end of the circular-table portion III, and a recess is provided on the circular-table portion IV.

Preferably, the atomizing core comprises a cannular portion, two heating coils fixed to a lower end of the cannular portion, the heating coils pass through the ceramic silicone sleeve to attach and connect to the bottom thereof; an opening is provided on an upper end of the ceramic silicone sleeve.

Preferably, the silicone cover is provided as T-shaped, a lower end thereof is arranged in the glass chamber; a plug is provided on an upper end of the silicone cover, a hard metal bracket is arranged between the silicone cover and the atomizing bracket, and the hard metal bracket is sleeved on the atomizing bracket; the atomizing bracket is provided with a lug boss to support the hard metal bracket; and a through hole adapted to the plug is provided on the silicone cover.

Preferably, the hard metal bracket is shaped as a handle comprising a through hole in the middle, and the through hole is adapted to the outer diameter of the atomizing bracket.

After adopting above structure, the invention has the following advantageous:

(1) Environmental protection: The invention has no objectional odor when in use, which solves the problem in the prior art that the plastic materials may deteriorate and emit an odor;

(2) High-temperature resistance: the glass atomizing core used for electronic cigarette in the invention can withstand the high temperature of more than 300° C., which solves the problem in the prior art that the plastic atomizing core of electronic cigarette may deteriorate and be soft at high temperature;

(3) Safety: the glass atomizing core used for electronic cigarette in the invention is made of advanced borosilicate glass material and with a thickness of 1.3 mm, according to the test result, which is not easily broken;

(4) Convenience: the glass atomizing core used for electronic cigarette in the invention has a firm casing, the smoke oil can be safety stored therein without being deformed by transportation, illumination, and vibration and extrusion in use, and the atomizing core is stored in the aluminum foil package, with tinfoil sealed, the atomizing core is clean and convenient to use; there is no atmospheric pressure in the package, which ensures no oil leakage, and solves the problem that the conventional plastic atomizing core may be deformed, even broken by vibration and extrusion in transportation, assembly or in use, causing the natural overflowing of the smoke oil;

(5) Materials saving: the glass atomizing core used for electronic cigarette in the invention has small volume, less materials and low cost, and is cheaper than soft plastic atomizing core, which solves the problem in the prior art that the atomizing cores of electronic cigarette need to be replaced frequently, and reduces resource consumption and consumer expenses;

(6) standard: the volume of the atomizing core of the invention is 0.8 ml, smaller than that of the prior plastic atomizing core, and 0.8 ml is the optimum volume of the daily consumption of an adult, thereby preventing the occurrence of the health problems caused by excessive daily smoking.

### BRIEF DESCRIPTION OF THE DRAWINGS

FIG. 1 shows the stereo structure of a glass atomizing core used for electronic cigarette;

FIG. 2 shows the top view of a glass atomizing core used for electronic cigarette;

FIG. 3 shows the sectional view of a glass atomizing core used for electronic cigarette;

FIG. 4 shows the explosive view of a glass atomizing core used for electronic cigarette.

### DESCRIPTION OF THE PREFERRED EMBODIMENTS

The invention will be further described in detail below with reference to accompanying drawings.

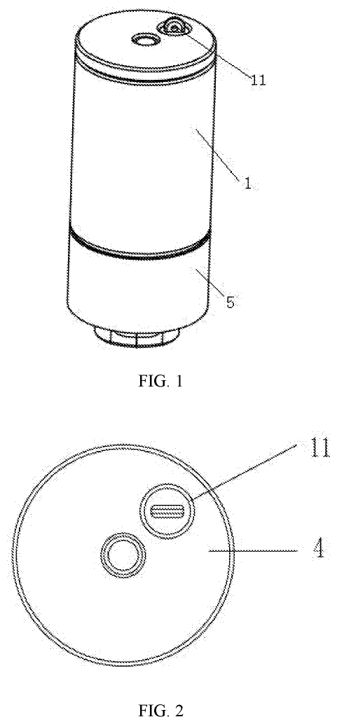

As shown in FIG. 1-4, a glass atomizing core used for electronic cigarette, comprising a glass chamber 1, an atomizing bracket 2 and an atomizing core 3 arranged in sequence from outside to inside, wherein an upper end of the glass chamber 1 is clamped with an adduction silicone cover 4, a lower end of the glass chamber 1 is arranged with a steel sleeve 5, and an electrode 6 is arranged on a lower end of the steel sleeve 5;

the glass chamber 1 is provided as a cannular shape, and a concave notch 101 is disposed on outer wall surface of a lower end thereof, a fixing ring 7 is clamped with the notch 101, and the fixing ring 7 is connected to the steel sleeve 5;

the atomizing core 3 is connected to a lower end of the atomizing bracket 2, an upper end of the atomizing core 3 is wound with nonwovens 8, and a lower end of the atomizing core 3 is sleeved with a ceramic silicone sleeve 9 and electrically connected with the electrode 6.

In the specific embodiment, after putting the invention into the main machine of electronic cigarette and fixing, and pushing the switch of the main machine, the battery output electric energy into the atomizing core 3, the ceramic of the atomizing core 3 may preheat (after about 10 s), the motor of the atomizing core 3 vibrating (to remind user the finish of preheating), the user can push the switch to operate the electronic cigarette to smoke, the airflow circulates in the atomizing core 3, the smoke oil is atomized by airflow and high temperature and is sucked into the user's mouth.

The plastic core of the prior art is replaced with the ceramic atomizing core 3 with the glass chamber 1, thereby the invention can be applied to the high temperature above 300° C. without deformation and deterioration; the glass chamber 1 is made of advanced borosilicate glass material and with a thickness of 1.3 mm, which is not easily broken; and taking the glass chamber 1 as a casing of the atomizing core 3, thereby safely storing the smoke oil inside to prevent vibration caused by transportation, illumination or use; moreover, the volume of the atomizing core 3 of the invention is 0.8 ml, which is the optimum volume of the daily consumption of an adult, thereby preventing heavy smoking. The working principle of the atomizing core 3 is prior art and will not be described herein.

As shown in FIG. 4, the glass chamber 1 comprises a cylindrical portion I 102 and a cylindrical portion II 103 arranged in sequence from top to bottom, the outer diameter of the cylindrical portion I 102 is larger than that of the cylindrical portion II 103, and the central axes of the two are collinear; a ring belt 1031 is arranged on the cylindrical portion II 103 to form the notch adapted to the fixing ring 7.

In the specific embodiment, the ring belt 1031 is an annular lug boss structure comprising a larger outer diameter than the cylindrical portion II 103, thereby achieving mutual clamping with the fixing ring 7.

A chamber adapted to the atomizing bracket 2 is vertically arranged inside the glass chamber 1, thereby the atomizing bracket 2 is accommodated therein; the fixing ring 7 is clamped with a lower end of the glass chamber 1 to form a clamping cannular body.

The fixing ring 7 comprises a half-cylinder fixing ring I 71 and a half-cylinder fixing ring II 72, a cylindrical fixing ring is composed of the fixing ring I 71 and the fixing ring II 72; a clamping portion adapted to the ring belt is disposed on the inner wall of the fixing ring I 71 and the fixing ring II 72, the clamping portion is provided as an annular groove structure.

Both the fixing ring I 71 and the fixing ring II 72 are provided with a curved belt with a certain height, and the two are combined into a ring belt which is sleeved on a lower end of the glass chamber 1. At least two bulges are provided on one side of the fixing ring I 71, a concave structure adapted to the bulges is provided on one side of the fixing ring II 72, thereby the fixing ring I 71 and the fixing ring II 72 are clamped with each other and fixed to the outside of the glass chamber 11, the bulges herein is provided as prismoids, and the concave structure is adapted to the prismoids.

A chamber adapted to the atomizing core 3 is provided in the atomizing bracket 2, the atomizing bracket 2 comprises a circular-table portion I 21, a circular-table portion II 22, and a circular-table portion III 23, which are arranged in sequence from top to bottom, the outer diameters of the circular-table portion I 21, the circular-table portion II 22 and the circular-table portion III 23 are gradually increased, a transition portion 24 is provided between the circular-table portion I 21 and the circular-table portion II 22, and the central axes of the circular-table portion I 21, the circular-table portion II 22, the circular-table portion III 23 and the transition portion 24 are collinear.

In the process of specific assembly, the upper end of the atomizing bracket 2 extends to the upper end of the glass chamber 1 and protrudes out of glass chamber 1, the silicone cover 4 is arranged on the upper end of the atomizing bracket 2, and a circular hole for inserting the upper end of the atomizing bracket 2 is provided on the silicone cover 4.

The circular-table portion II is provided with a groove 221 at intervals, an O-shaped ring 10 is provided in the groove 221, and an outer portion of the O-shaped ring 10 is attached and connected to the lower end of the glass chamber 1.

The arrangement of the O-shaped ring 10 facilitates the tight connection between the glass chamber 1 and the atomizing bracket 2, thereby improving the tightness of the connection between the two.

The chamber is arranged in the circular-table portion II 22 and the circular-table portion III 23.

A chamber is provided for accommodating the atomizing core 3 inside the atomizing bracket 2; and a transition portion 24 is provided for preventing the atomizing core 3 from moving upward in use, and the maximum inner diameter of the transition portion 24 is smaller than the maximum outer diameter of the atomizing core 3, thereby the atomizing core 3 is restrained at the lower end position of the atomizing bracket 2 to improve the stability of the invention.

The atomizing core 3 comprises a a cannular portion 31, two heating coils 32 fixed to a lower end of the cannular portion 31, the heating coils 32 is connected into the ceramic silicone sleeve 9 to attach and connect to the bottom thereof; and an opening is provided on an upper end of the ceramic silicone sleeve 9.

The ceramic silicone sleeve 9 is externally connected to the atomizing bracket 2 and pressed against the O-shaped ring 10 to further improve the tightness of the invention.

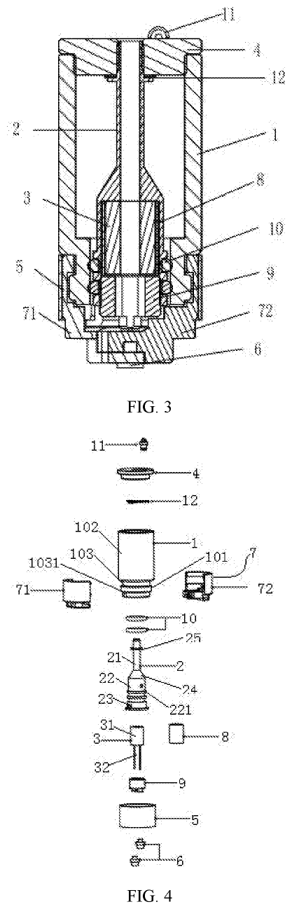

As shown in FIG. 3, the silicone cover 4 is provided as T-shaped, a lower end thereof is arranged in the glass chamber 1; a plug 11 is provided on an upper end of the silicone cover 4, a hard metal bracket 12 is arranged between the silicone cover 4 and the atomizing bracket 2, and the hard metal bracket 12 is sleeved on the atomizing bracket 2; the atomizing bracket 2 is provided with a lug boss 25 to support the hard metal bracket 12; and a through hole adapted to the plug 11 is provided on the silicone cover 4.

The hard metal bracket 12 is provided as a handle comprising a through hole in the middle, and the through hole is adapted to the outer diameter of the atomizing bracket 2.

In the specific use of the invention, the silicone plug 11 can be directly pulled out, and is covered when not in use, which is much more convenient; the arrangement of the hard metal bracket 12 is convenient to realize the further fixing of the atomizing bracket;

The lug boss 25 is provided as a cylindrical structure comprising a larger outer diameter than the circular-table portion I 21 to support the hard metal bracket 12 sleeved on the circular-table portion I 21.

1. A glass atomizing core used for electronic cigarette, comprising a glass chamber, an atomizing bracket and an atomizing core arranged in sequence from outside to inside, wherein an upper end of the glass chamber is clamped with an adduction silicone cover, a lower end of the glass chamber is arranged with a steel sleeve, and an electrode is arranged on a lower end of the steel sleeve;
the glass chamber is provided as a cannular shape, and a concave notch is disposed on outer wall surface of a lower end thereof, a fixing ring is clamped with the notch, and the fixing ring is connected to the steel sleeve;
the atomizing core is connected with a lower end of the atomizing bracket, an upper end of the atomizing core is wound with nonwovens, and a lower end of the atomizing core is sleeved with a ceramic silicone sleeve and electrically connected with the electrode.

2. The glass atomizing core used for electronic cigarette of claim 1, wherein the glass chamber comprises a cylindrical portion I and a cylindrical portion II arranged in sequence from top to bottom, the outer diameter of the cylindrical portion I is larger than that of the cylindrical portion II, and the central axes of the two are collinear; a ring belt is arranged on the cylindrical portion II to form the notch adapted to the fixing ring.

3. The glass atomizing core used for electronic cigarette of claim 2, wherein the fixing ring comprises a half-cylinder fixing ring I and a half-cylinder fixing ring II, a cylindrical fixing ring is composed of the fixing ring I and the fixing ring II; a clamping portion adopted to the ring belt is disposed on the inner wall of the fixing ring I and the fixing ring II, the clamping portion is provided as an annular groove structure.

4. The glass atomizing core used for electronic cigarette of claim 1, wherein a chamber adopted to the atomizing core is provided in the atomizing bracket, the atomizing bracket comprises a circular-table portion I, a circular-table portion II, and a circular-table portion III, which are arranged in sequence from top to bottom, the outer diameters of the circular-table portion I, the circular-table portion II and the circular-table portion III are gradually increased, a transition portion is provided between the circular-table portion I and the circular-table portion II, and the central axes of the circular-table portion I, the circular-table portion II, the circular-table portion III and the transition portion are collinear.

5. The glass atomizing core used for electronic cigarette of claim 4, wherein the circular-table portion II is provided with a groove at intervals, an O-shaped ring is provided in the groove, and an outer portion of the O-shaped ring is attached and connected with the lower end of the glass chamber; and a through hole is provided on the circular-table portion II.

6. The glass atomizing core used for electronic cigarette of claim 4, wherein the chamber is arranged in the circular-table portion II and the circular-table portion III.

7. The glass atomizing core used for electronic cigarette of claim 4, wherein a circular-table portion IV is arranged on a lower end of the circular-table portion III, and a recess is provided on the circular-table portion IV.

8. The glass atomizing core used for electronic cigarette of claim 1, wherein the atomizing core comprises a cannular portion, two heating coils fixed to a lower end of the cannular portion, the heating coils pass through the ceramic silicone sleeve to attach and connect to the bottom thereof; an opening is provided on an upper end of the ceramic silicone sleeve.

9. The glass atomizing core used for electronic cigarette of any one of claim 1-8, wherein the silicone cover is provided as T-shaped, a lower end thereof is arranged in the glass chamber; a plug is provided on an upper end of the silicone cover, a hard metal bracket is arranged between the silicone cover and the atomizing bracket, and the hard metal bracket is sleeved on the atomizing bracket; the atomizing bracket is provided with a lug boss to support the hard metal bracket; and a through hole adapted to the plug is provided on the silicone cover.

10. The glass atomizing core used for electronic cigarette of claim 9, wherein the hard metal bracket is shaped as a handle comprising a through hole in the middle, and the through hole is adapted to the outer diameter of the atomizing bracket.
